(19) United States
(12) Patent Application Publication
(10) Pub. No.: US 2007/0197960 A1
(43) Pub. Date: Aug. 23, 2007

(54) Multiple septum cartridge for medication dispensing device

(76) Inventors: Kenneth Alan Ritsher, Chicago, IL (US); Anastasios G. Karahalios, Lincolnwood, IL (US)

Correspondence Address: ELI LILLY & COMPANY, PATENT DIVISION, P.O. BOX 6288, INDIANAPOLIS, IN, 46206-6288, US

(21) Appl. No.: 11/572,430

(22) PCT Filed: Jul. 26, 2005
(86) PCT No.: PCT/US05/26399

§ 371 Date: Jan. 22, 2007

Related U.S. Application Data
(60) Provisional application No. 60/591,570, filed on Jul. 27, 2004.

(51) Int. Cl. A61M 31/00

(52) U.S. Cl. 604/93.01; 604/82

(57) ABSTRACT

A cartridge for a medication dispensing device which includes multiple septums. The cartridge includes a body and an axially shiftable piston in fluid-tight engagement with an interior surface of the body. The first septum covers a dispensing port that opens to an interior hollow of the body at a proximal location. The second septum covers a filling port that opens to the interior hollow. The first septum is arranged to be engaged by a dispensing conduit moving generally perpendicular to the axial direction for fluidly communicating with the interior hollow during a dispensing operation of the device. The second septum is arranged to be engaged by a filling conduit for fluidly communicating with the interior hollow during filling of the cartridge.

### BACKGROUND OF THE INVENTION

The present invention pertains to medication dispensing devices, and, in particular, to a cartridge for a portable medication dispensing device.

To allow a person to conveniently and accurately administer medicine, a variety of differently configured devices, notably including a class of devices known as injection pens, have been developed. Frequently these devices are equipped with a cartridge including a piston and containing a multi-dose quantity of liquid medication. A drive member of the device is shiftable forward to advance the piston away from one end of the cartridge to dispense the contained medication from an outlet at the opposite cartridge end, such as through a needle that penetrates a stopper or septum at that opposite end.

A variety of cartridge designs are known and have been developed to address certain requirements of their use. For example, cartridges disclosed in WO 03/030810 are intended to limit wastage of pharmaceuticals within the cartridge. Many known cartridges are pre-filled by the manufacturer such that a user merely needs to install such a cartridge into the dispensing device for use. However, as such pre-filled cartridges naturally need to be manufactured, shipped and stored until use, the amount of time that the medicine resides within the cartridge may necessitate use of certain materials of cartridge construction which are known to be suitable for the pharmaceuticals contained therein. This suitability requirement may discourage the use of certain plastic materials for the cartridge as, for example, the long-term stability of the pharmaceuticals in such materials may not be known. And, while providing a user with a pharmaceutical in, for example, a standard glass container for filling into the, for example, plastic cartridge may avoid long term exposure of the pharmaceutical to the plastic, such filling may introduce its own problems, such as possibly compromising the function of the cartridge or being too inconvenient for a user.

Thus, it would be desirable to provide an apparatus that overcomes one or more of these and other shortcomings of the prior art.

### BRIEF SUMMARY OF THE INVENTION

In one form thereof, the present invention provides a cartridge for use in a medication dispensing device including a dispensing needle. The cartridge includes a body having an interior hollow, a piston in fluid-tight engagement with an interior surface of the body to seal a distal end of the interior hollow, the piston being shiftable in an axial direction within an axially extending length portion of the body, wherein the piston is shifted from a distal position to a proximal position in the axial direction during a dispensing operation, a first sealing member covering a dispensing port that opens to the interior hollow, and a second sealing member covering a filling port that opens to the interior hollow. The first sealing member has a puncturing area arranged to be penetrated by the dispensing needle moving generally perpendicular to the axial direction into the dispensing port during a dispensing operation of the device. The second sealing member has a puncturing area for penetration by a filling needle moving into the filling port during filling of the cartridge.

One advantage of the present invention is that a medication cartridge may be provided which utilizes different openings for filling the cartridge and for emptying the cartridge during its use in a medication dispensing device, thereby allowing the septums for such openings to be particularly adapted, such as by having different penetration requirements or sealing properties, for use with different sized filling and dispensing needles or the like.

Another advantage of the present invention is that a medication cartridge may be provided which is easily fillable by a user for use in a medication dispensing device.

Still another advantage of the present invention is that a medication cartridge may be provided with a simple and cost-effective form.

### BRIEF DESCRIPTION OF THE DRAWINGS

The above-mentioned and other advantages and objects of this invention, and the manner of attaining them, will become more apparent, and the invention itself will be better understood, by reference to the following description of embodiments of the invention taking in conjunction with the accompanying drawings, wherein:

FIG. 1 is a perspective view of a first embodiment of a cartridge of the present invention for use with a medication dispensing device, which cartridge is arranged in a ready-to-be-filled state;

FIG. 2 is an exploded perspective view of the cartridge of FIG. 1;

FIG. 3 is an axial cross-sectional view of the cartridge of FIG. 1;

FIG. 4 is an axial cross-sectional view similar to the view of FIG. 3, but after the cartridge has been filled;

FIG. 5 is a view of portions of the cartridge of FIG. 3, diagrammatically showing a filling needle during cartridge filling;

FIG. 6 is an end view of the cartridge of FIG. 4, diagrammatically showing one suitable dispensing needle of a medication dispensing device poised for penetrating the dispensing septum;

FIG. 7 is a view of portions of the cartridge of FIG. 4, diagrammatically showing the dispensing needle of FIG. 6 during cartridge dispensing;

FIG. 8 is an exploded perspective view of a second embodiment of a cartridge of the present invention for use with a medication dispensing device;

FIG. 9 is an axial cross-sectional view of the assembled cartridge of FIG. 8;

FIG. 10 is a perspective view of still another embodiment of a cartridge of the present invention;

FIG. 11 is an exploded perspective view of still another embodiment of a cartridge of the present invention;

FIG. 12 is an axial cross-sectional view of the proximal portion of the assembled cartridge of FIG. 11;

FIG. 13 is an exploded perspective view of still another embodiment of a cartridge of the present invention;

FIG. 14 is an exploded perspective view of still another embodiment of a cartridge of the present invention; and

FIG. 15 is a perspective view of the assembled cartridge of FIG. 14.

Corresponding reference characters indicate corresponding parts throughout the several views. Although the drawings represent embodiments of the present invention, the drawings are not necessarily to scale, and certain features may be exaggerated or omitted in some of the drawings in order to better illustrate and explain the present invention.

### DETAILED DESCRIPTION OF THE INVENTION

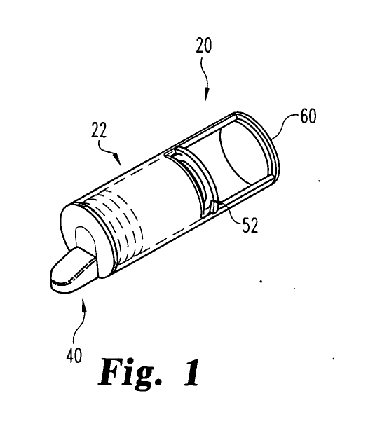

Referring now to FIG. 1, there is shown a first embodiment of a medication cartridge of the present invention. The cartridge, generally designated 20, is intended for use in a dispensing device that is portable by a user to discreetly administer a dose of medication. Although described herein as used with an injection device, it will be appreciated that the cartridge of the present invention may find beneficial application with other devices including infusion apparatus such as insulin pumps, in which pumps the medication may be dispensed from the cartridge automatically, as opposed to the more conscious delivery by a user utilizing conventional injection devices, such as injection pens and the like.

Cartridge 20 includes a body 22 shown having a cylindrical, tubular portion or barrel 24, a disc portion 26, and a hollow, proximally close-ended protuberance 28. Body 22 is formed of a medically and mechanically suitable material, such as a polymer or blend of polymer materials, such as a plastic, to facilitate its manufacture and accuracy in sizing in production. Although shown as having a unitary construction, body 22 may be sealingly assembled during manufacture from multiple complementarily shaped and sized pieces.

Body disc portion 26 seals off the proximal end of barrel 24 and thereby the barrel interior hollow, and includes a first opening 30 therethrough and a radially offset, second opening 31 therethrough. Body opening 30 is centrally located and thereby aligned with the axis of barrel 24, and serves as a filling port of the cartridge. Body opening 31 leads to an internal hollow portion 33 within protuberance 28. An opening 35 in a radially inwardly, or laterally inwardly, facing surface 37 of protuberance 28 serves as a dispensing port of the cartridge.

Protuberance 28 projects proximally from body disc portion 26 within the circular profile extended of barrel 24. Surface 37 is shown oriented at an acute angle relative to the central axis of barrel 24, which slight angling allows additional space within an associated dispensing device to accommodate a needle assembly, positioned thereabove from the perspective of a FIG. 6 viewer, for which cartridge 20 is well suited. In other embodiments in which a dispensing port of the protuberance is radially offset as shown is employed, different orientations of surface 37, including different anglings or surface 37 being parallel to the barrel central axis, may be provided if such accommodate a piercing needle that moves generally perpendicular to the cartridge axis during septum penetration. Still further, protuberance 28 need not be radially offset but rather could be centrally located, such as if the filling port was radially offset or was provided in the protuberance, such as at its proximal end.

A cup-shaped, resilient sealing element 40 fits over a portion of the body 22 to provide fluid tight seals for body openings 30 and 35. Sealing element 40 includes a pocket portion 42, and an arch-shaped flange portion 44 having an axially protruding annular plug 45. Seal pocket portion 42 fits over protuberance 28. Seal flange portion 44 overlays the center of disc portion 26 and fits within an arch-shaped recess formed into the proximal face of disc portion 26, with the plug 45 of flange portion 44 closely inserting into body opening 30.

Sealing element 40 is attached to body 22 in a suitable fashion to ensure that pocket portion 42 seals with protuberance 28 to provide a fluid tight seal or septum for opening 35, and that flange portion 44 seals with disc portion 26 to provide a separate fluid tight seal or septum for opening 30. Pocket portion 42 need not cover all of protuberance 28, but rather merely needs to be large enough to be sealingly secured around opening 35. The attachment of sealing element 40 and body 22 may be by any suitable means known in the art, including by overmolding or co-molding sealing element 40 with body 22.

Sealing element 40 is made from a suitable material or combination of materials, such as polyisoprene or butyl rubber, and may be formed with different physical characteristics to provide appropriate sealing properties in view of the conduits expected to temporarily access the body openings sealingly covered thereby. For example, to account for a filling needle expected to insert into opening 30 which has a larger gauge than a dispensing needle expected to insert into opening 35, the flange portion 44, or at least the area thereof expected to be penetrated by such filling needle, may be formed with a thicker dimension or a different combination of materials than pocket portion 42, or at least the area thereof expected to be penetrated by such dispensing needle.

Body barrel 24, along its internal piston engaging length, has a constant inner diameter that refers to the fact that its associated piston maintain its seal, as naturally the tubular portion inner diameter may have slight variations, such as a draft angle or surface inconsistencies from manufacture. Body barrel 24 includes a notch 48 distally of its piston engaging length which allows the installed piston to be accessed by a piston driving assembly of a dispensing device for which cartridge 20 is particularly suited, such as a device disclosed more fully in U.S. Provisional Patent Application No. 60/558,412, which is incorporated herein by reference.

Body barrel 24 defines a medicine-fillable, variable volume hollow or reservoir 50 that is completely closed at its distal end by a piston, generally designated 52, that can travel along the axis of the cartridge. The medicine fillable interior of the body may be vacuum evacuated during manufacture. Piston 52 of cartridge 20 includes a cylindrical body 54, made of a suitable material such as plastic, with a closed proximal face 55 and which is ringed by at least one 0-ring 56, such as the two rings shown, that seats within a circumferential groove in body 54. 0-rings 56 allow piston 52 to be axially slidably and sealably engaged with the interior wall of body barrel 24 to hold medication, such as insulin or another therapeutic, within reservoir 50. Slot 58 near the piston body distal end receives a driver of the piston driving assembly of the dispensing device. Alternate piston constructions, including alternate cross-sectional shapes for use with correspondingly different cross-sections of the body tubular portion or barrel, may be employed within the scope of the invention.

A plastic end cap 60 is shown attached to the distal end of body barrel 24. End cap 60 is fixed to the body during manufacture and helps to prevent axially loads from being inadvertently placed on the distal face of the piston 52 when the cartridge is being handled outside the dispensing device.

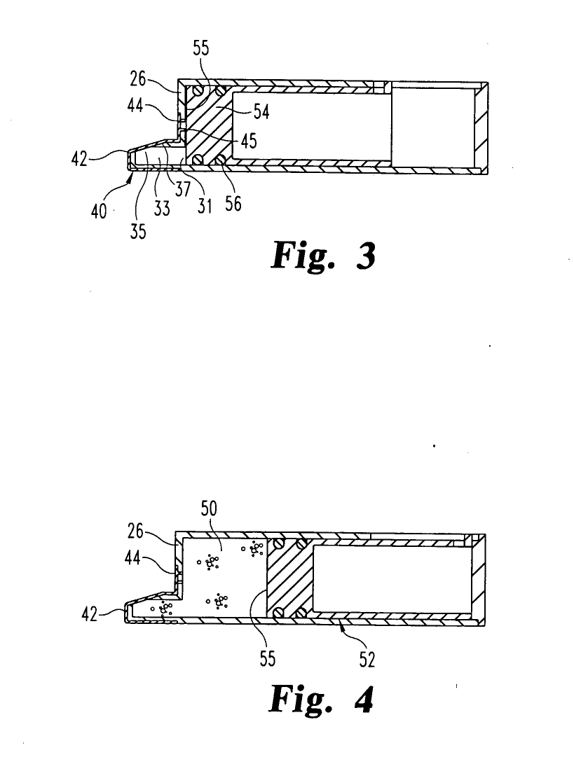

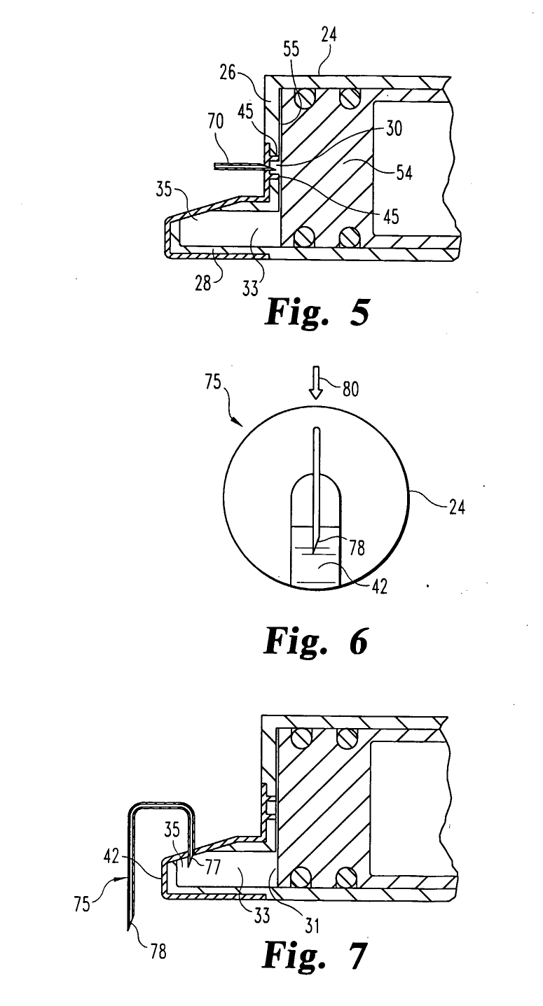

The structure of cartridge 20 will be understood further in view of the following general explanation of one form of its operation. Cartridge 20 is provided to its intended user in an unfilled state, such as with the arrangement of its components being as shown in FIG. 3. Prior to loading cartridge 20 into its associated dispensing device, and as diagrammatically shown in FIG. 5, the user inserts a filling needle 70 that penetrates or pierces the penetration area of seal flange portion 44 to enter the central space of annular plug 45 so as to enter the space within cartridge body opening 30.

The apparatus including the filling needle 70 is preferably constructed in view of the dimensions and shape of cartridge 20, such that the inserted tip of filling needle 70, when axial insertion in the distal direction is halted due to the engagement of the filling apparatus with the cartridge 20, does not reach the plane of the distal face of disc portion 26 and thereby does not contact or penetrate into piston end face 55.

Then, as the user manipulates the filling apparatus to eject medication contained in the filling apparatus through needle 70 and into the reservoir 50 of body 22, the inflowing medication forces piston 52 distally within body 22 by hydraulic pressure. During this filling time, the medication also automatically passes through body opening 31 to fill the body interior hollow portion 33 within protuberance 28. Filling is complete when, for example, the filling apparatus has been emptied, such as in the case of where cartridge 20 is adapted to hold the contents transferred from a standard 3 ml glass cartridge known in the art, which standard cartridge may be made part of or loaded in the filling apparatus. Then, filling needle 70 is axially removed proximally, whereby the septum formed by seal flange portion 44, due to its resiliency and design, reseals itself to prevent fluid escape from the reservoir 50.

After filling, cartridge 20 is configured as shown in FIG. 4. Then, the user loads the filled cartridge 20 into the associated dispensing device for use therein. When that dispensing device is subsequently operated in order to dispense medication, the device shifts a dispensing needle 75 of that device in a direction, indicated at arrow 80, generally perpendicular to the axis of the cartridge. As needle 75 is so shifted, a tip 77 of needle 75 penetrates or pierces the penetration area of seal pocket portion 42 so as to enter the space within cartridge body opening 35, and possibly hollow portion 33, at a location proximal of the most proximal position to which piston 52 is shiftable during use, to provide a fluid flow outlet by which medicine within cartridge 20 can be dispensed. In the shown embodiment, during the septum piercing travel of needle 75, an injecting portion 78 of needle 75 which is parallel to and extends farther than the needle tip 77 projects from the device to penetrate a user's skin that has been placed against the exterior of the dispensing device in which cartridge 20 is disposed.

In FIG. 6, dispensing needle 75 is shown prior to having been plunged to penetrate the dispensing septum. In FIG. 7, dispensing needle 75 is shown after having been plunged to pierce the dispensing septum.

Although shown as having an inverted U-shaped design with a septum piercing leg that is shorter than its user piercing leg, dispensing needle 75 is merely illustrative and not intended to be limiting. For example, instead of a parallel leg 78, the dispensing needle could have a downstream end connected to another conduit that leads to, for example, an injection or infusion needle.

When dispensing needle 75 is arranged as shown in FIG. 7, as dispensing device is operated to advance cartridge piston 52 proximally within body barrel 24, medication is forced from the main portion of the reservoir 50 through opening 31 and the interior hollow 33 that forms a portion of the reservoir, and out through the dispensing needle 75.

After use of the medication dispensing device, dispensing needle 75 may be automatically retracted in the direction perpendicular to the cartridge axis back to the position shown in FIG. 6, whereby the septum formed by seal pocket portion 42, due to its resiliency and design, reseals itself to prevent fluid escape from reservoir 50, and the dispensing needle may be replaced with another identical needle that is indexed into place automatically or otherwise by the dispensing device.

Medication cartridge 20 can continue to be used until, for example, the proximal face 55 of piston 52 abuts the distal face of disc portion 26, at which time no additional medication can be forced from the reservoir. It will be appreciated that at such time the cartridge piston 52 is in its entirety still located distally of dispensing port 35. At such time, the disposable cartridge 20 can be removed from the dispensing device, if such device is reuseable, and discarded and then replaced with a similar, user filled replacement cartridge 20. In situations where the dispensing device is intended to be disposable, after cartridge 20 has been manipulated to the arrangement as shown in FIG. 3, the entire dispensing device including the spent cartridge may be discarded.

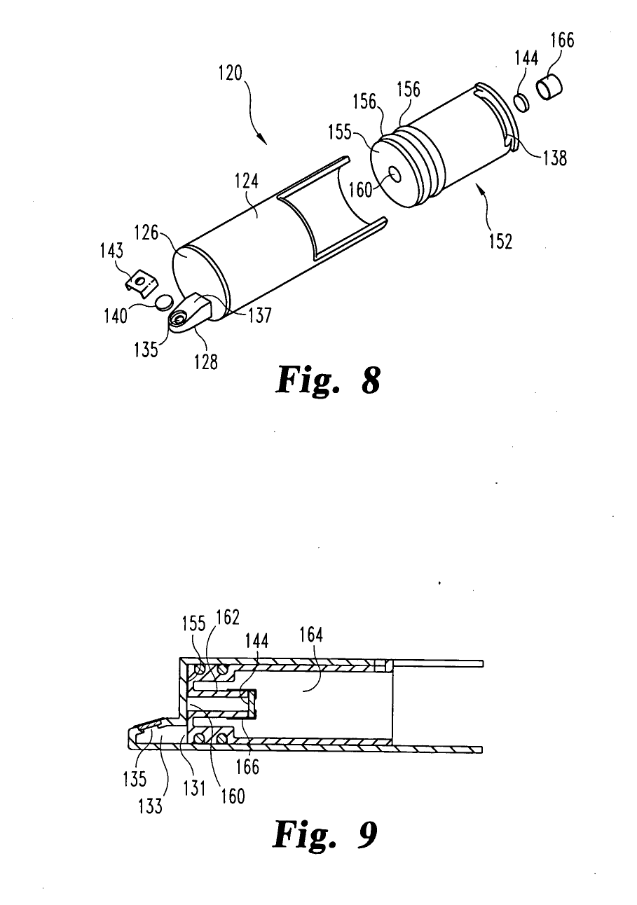

Referring now to FIG. 8, there is shown an alternate embodiment of a multiple septum cartridge of the present invention for use with a dispensing device having a transversely plunging dispensing needle. This cartridge, generally designated 120, is substantively similar to cartridge 20 in most material respects, and includes a barrel 124, a disc portion 126, and a protuberance 128. Disc portion 126 includes an offset opening 131 leading to an internal hollow portion 133 within protuberance 128. An opening 135 of protuberance 128 serves as a dispensing port of the cartridge.

A circular, resilient sealing element 140 fits within a recess in the generally laterally facing surface 137 of protuberance. Sealing element 140 is held within the recess by an apertured metal plate 143 that is crimped to protuberance 128, thereby providing a fluid tight septum for opening 135. The crimping plate aperture allows free passage of the dispensing needle.

Cartridge piston 152 closes off the distal end of a medicine-fillable, variable volume reservoir 150 and includes a cylindrical body 154. O-rings 156 seat within circumferential grooves that ring body 154 for the piston to be axially slidably and sealably engaged with the interior wall of the cartridge barrel 124. Slot 158 cooperates with the piston driving assembly of the dispensing device.

The proximal face 155 of piston body 154 has a central opening 160 that extends within a cylindrical tube 162 integrally formed with body 154. Tube 162 distally projects into an interior hollow 164 of body 154. The distal end of tube 162 is sealingly capped by a disc-shaped sealing element 144 that is secured by a metal sleeve 166 crimped to the exterior of tube 162 to thereby provide a septum for the tube opening 160.

A filling of cartridge 120 may be performed with a filling needle and its associated apparatus being proximally inserted into piston body hollow 164. The filling needle tip can pass through an end aperture in crimping sleeve 166, through the penetration area of sealing element 144, and into the space within opening 160 for the introduction of medication into the cartridge.

No plastic end cap is attached to the distal end of body barrel 124 as such, unless differently designed from cap 60, would interfere with the cartridge filling described above.

Referring now to FIG. 10, there is shown still another alternate embodiment of the multiple septum cartridge of the present invention. Cartridge 220 is similar to cartridge 20, but includes a keying 225 on its body barrel 224 to cause cartridge 220 to be properly oriented when installed within a complementarily designed cavity of the dispensing device.

Cartridge 220 includes an L-shaped protuberance 228 that extends from body disc portion 226. A first leg 229 of protuberance 228 includes a filling opening aligned with a central opening in disc portion 226, which filling opening is sealed by a circular, resilient sealing element 235 that seats within a recess in first leg 229 and is held thereat by a crimping ring 236. The hollow main portion 230 of protuberance 228 is radially offset and includes a dispensing port in a laterally facing surface, which dispensing port is sealed by a circular, resilient sealing element 240 that seats within a recess in main portion 230 and is held thereat by a crimping ring 242.

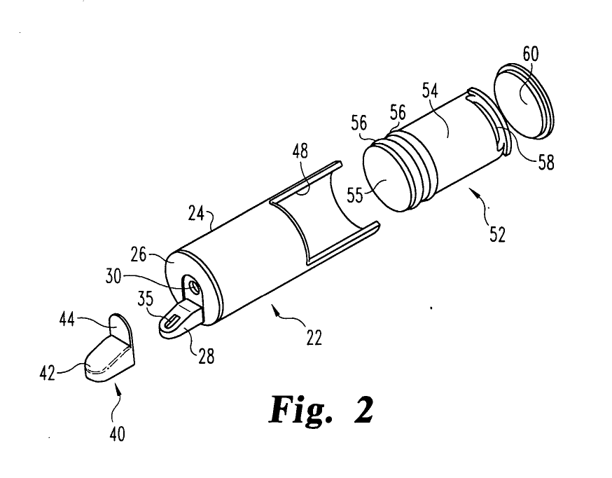

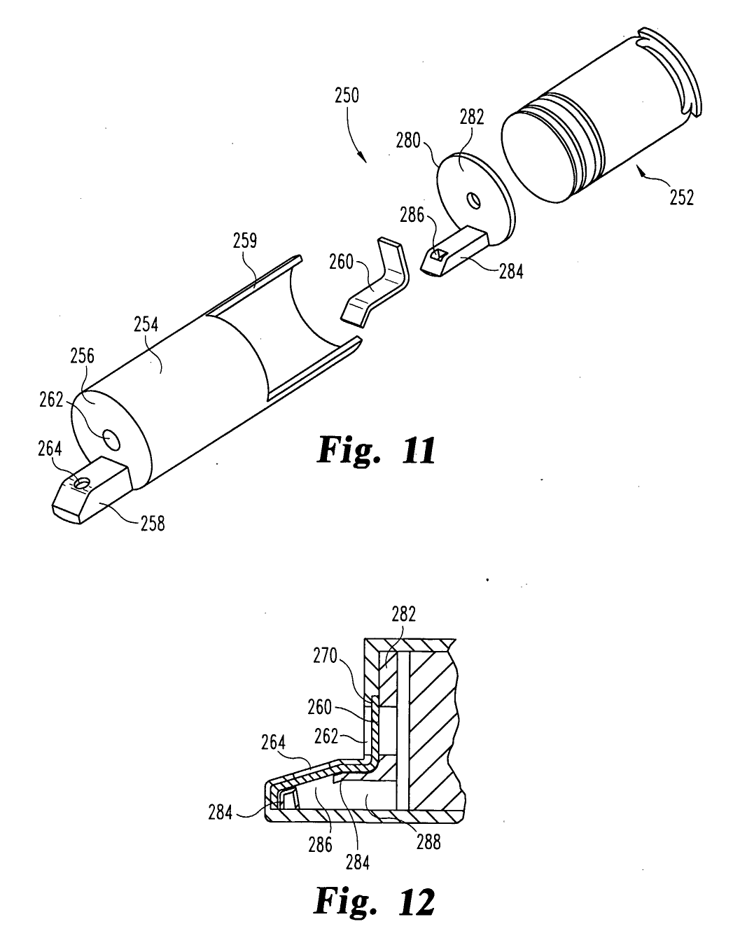

Referring now to FIGS. 11 and 12, there is shown still another alternate embodiment of the multiple septum cartridge of the present invention. The cartridge 250 includes a piston, generally designated 252, that is identical to piston 52 of the embodiment of FIG. 2. Cartridge 250 includes a barrel 254 with notch 259, a disc portion 256, and a protuberance 258. Sealing element 260 installs within the cartridge body and fits within a recess 270 formed in the interior surface of disc portion 256. Sealing element 260 is secured in place by a plastic plug member 280 including a centrally apertured, disc-shaped portion 282, and a nose portion 284 having an opening 286 that accesses the hollow interior 288 of nose portion 284. Plug portion 280 secures sealing element 260 in its proper, sealing arrangement that provides a fluid tight seal for openings 262 and 264, and plug portion 280 is held within the cartridge body in a suitable manner, such as a wedge or force fit therein, or alternatively with a suitable fastening such as an ultrasonic welding.

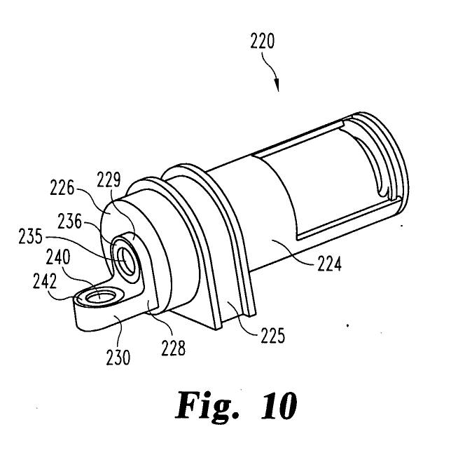

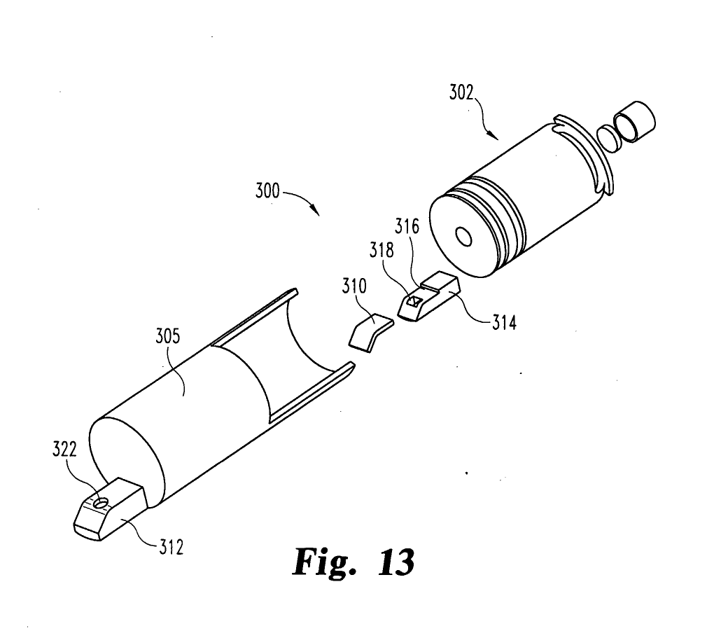

Referring now to FIG. 13, there is shown another alternate embodiment of the multiple septum cartridge of the present invention. The cartridge 300 includes a piston, generally designated 302, that is identical to piston 152 of the embodiment of FIGS. 8 and 9. The cartridge body is identical to that of the embodiment of FIG. 11, but lacks a central opening and internal recess in the disc portion. Sealing element 310 is wedged within the protuberance 312 via a plug portion 314 that has a recessed surface 316 to accommodate the sealing element 310. The recessed surface 310 includes an opening 318 therethrough accessing the not shown hollow interior of the plug portion 314, which interior is in communication with the internal reservoir of the cartridge barrel 305. Sealing element 310 is secured via plug portion 314 to seal opening 322.

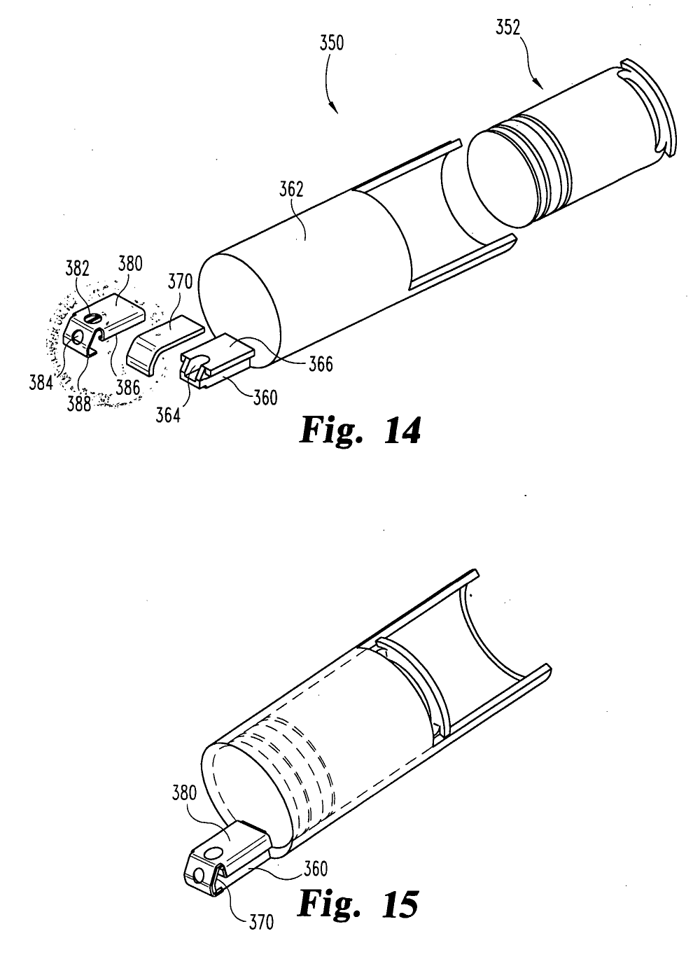

Referring now to FIGS. 14 and 15, there is shown still another alternate embodiment of a multiple septum cartridge of the present invention. The cartridge 350 includes a piston 352 identical to piston 252 of the embodiment of FIGS. 11 and 12. The cartridge protuberance 360 that proximally projects from the tubular barrel 362 includes an opening 364 at its proximal end that accesses the hollow interior of protuberance 360, which opening 364 extends within both the radially inward and proximal faces of the protuberance. The protuberance 360 is formed with a ledge 366. Sealing element 370 fits over protuberance 360 and is secured thereon via a crimping plate 380, having an injection opening 382 and a filling opening 384, which crimping plate 380 has flanges 386 and 388 that during assembly are respectively folded over the edges of ledge 366 and the forward edge of protuberance 360. In this embodiment, a single body opening serves as the filling port and the dispensing port, which single opening can be accessed in the axially and transverse directions.

While this invention has been shown and described as having preferred designs, the present invention may be modified within the spirit and scope of this disclosure. For example, one or both of the cartridge septums, rather than being provided in the form of a needle penetrable membrane, may be provided in the form of a valve that automatically opens when a mating portion of a filling conduit or a dispensing conduit, as appropriate, is brought into contact therewith. This application is therefore intended to cover any variations, uses or adaptations of the invention using its general principles. Further, this application is intended to cover such departures from the present disclosure as come within known or customary practice in the art to which this invention pertains.

We claim:

1. A cartridge for use in a medication dispensing device including a dispensing needle, the cartridge comprising:
a body having an interior hollow;
a piston in fluid-tight engagement with an interior surface of said body to seal a distal end of said interior hollow, said piston shiftable in an axial direction within an axially extending length portion of said body, wherein said piston is shifted from a distal position to a proximal position in the axial direction during a dispensing operation;
a first sealing member covering a dispensing port that opens to said interior hollow;
a second sealing member covering a filling port that opens to said interior hollow;
said first sealing member having a puncturing area arranged to be penetrated by the dispensing needle moving generally perpendicular to said axial direction into the dispensing port during a dispensing operation of the device; and
said second sealing member having a puncturing area for penetration by a filling needle moving into the filling port during filling of the cartridge.

2. The cartridge of claim 1 wherein said second sealing member puncturing area is arranged at an angle relative to said first sealing member puncturing area.

3. The cartridge of claim 2 wherein said second sealing member puncturing area is arranged perpendicular to said axial direction.

4. The cartridge of claim 1 wherein said first sealing member and said second sealing member are formed together as a single piece.

5. The cartridge of claim 1 wherein said filling port comprises a passage through said piston, whereby said second sealing member is attached to said piston to sealingly cover said passage.

6. The cartridge of claim 1 wherein said filling port comprises an opening in said body distinct from an opening in said body that serves as said dispensing port.

7. A cartridge for use in a medication dispensing device including a dispensing conduit, the cartridge comprising:
a body having an interior hollow;
a piston in fluid-tight engagement with an interior surface of said body to seal a distal end of said interior hollow, said piston shiftable in an axial direction within an axially extending length portion of said body, wherein said piston is shifted proximally in the axial direction during dispensing operations;
a first sealing member covering a dispensing port that opens to said interior hollow;
a second sealing member covering a filling port that opens to said interior hollow;
said first sealing member having an area arranged to be engaged by the dispensing conduit moving generally perpendicular to said axial direction for fluidly communicating with the interior hollow during a dispensing operation of the device; and
said second sealing member having an area arranged to be engaged by a filling conduit for fluidly communicating with the interior hollow during filling of the cartridge.
